(19) United States
(12) Patent Application Publication
(10) Pub. No.: US 2026/0185090 A1
(43) Pub. Date: Jul. 2, 2026

(54) Methods for Deprotecting and Purifying Oligonucleotide Compounds

(71) Applicant: Ionis Pharmaceuticals, Inc., Carlsbad, CA (US)

(72) Inventor: Dana Chreng, San Marcos, CA (US)

(73) Assignee: Ionis Pharmaceuticals, Inc., Carlsbad, CA (US)

(21) Appl. No.: 19/131,179

(22) PCT Filed: Nov. 21, 2023
(86) PCT No.: PCT/US2023/080762

§ 371 Date: May 19, 2025

Related U.S. Application Data
(60) Provisional application No. 63/384,658, filed on Nov. 22, 2022.

(51) Int. Cl. C12N 15/113

(52) U.S. Cl. CPC C12N 15/113; C12N 2310/11; C12N 2310/315; C12N 2310/321; C12N 2310/322; C12N 2310/3341; C12N 2310/351

(57) ABSTRACT

The present disclosure provides methods for deprotecting and purifying an oligomeric compound. The method provides for deprotection of a protecting group, which may be a trityl protecting group, and removal of a byproduct by diafiltration. Also provided are oligomeric compounds prepared by the method.

### FIELD

The present disclosure provides methods for deprotecting and purifying an oligomeric compound.

### BACKGROUND

Antisense technology is an effective means for modulating the expression of one or more specific gene products and can therefore prove to be uniquely useful in a number of therapeutic, diagnostic, and research applications. Chemically modified nucleosides may provide improvement of one or more properties, such as nuclease resistance, pharmacokinetics, or affinity for a target nucleic acid. Conjugate groups may be appended to a modified oligonucleotide to improve uptake into cells and/or tissues of interest.

Oligomeric compounds comprising an oligonucleotide and at least one conjugate group are chemically synthesized in a multi-step process that has the potential to introduce a number of unwanted contaminants. However, large-scale synthetic processes can incur prohibitive expense. Inefficient methods, and those that require a high number of steps, add difficulty and reduce margins for error. On production scale, such difficulty and expense can limit delivery of important medicines. The purification of oligomeric compounds remains an important challenge in bringing oligonucleotide-based therapeutics to patients, and improved synthetic and purification methods are needed.

### SUMMARY

Provided herein is a novel process for the deprotection and isolation of protected, and in particular trityl-protected, oligomeric compounds using tangential flow filtration (TFF). The protected oligomeric compound is deprotected in a retentate vessel. The deprotection reaction proceeds by addition of a deprotection reagent such as an acid (e.g., glacial acetic acid) to a solution of the oligomeric compound. Reaction temperature may be controlled, as well as reaction time. In a subsequent step, a deprotection byproduct (e.g., a trityl alcohol) is removed by diafiltration against a solution containing a buffer and an alcohol (e.g., methanol) and optionally a salt. The solution is formulated to keep both the deprotected oligomeric compound and deprotection byproduct in solution. In a subsequent step, a buffer exchange is performed in order to isolate the deprotected oligomeric compound in the solution of choice for further downstream processing. For example, the solution can be diafiltered with purified water (thereby removing alcohol and optional salt) and then isolated, e.g., by freeze drying. Alternatively, the oligonucleotide solution can be diafiltered with salt buffer in preparation for further synthetic steps.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “protected oligomeric compound solution” means a solution that carries an oligomeric compound in a flow filtration system.

As used herein, “deprotection solution” means a solution that carries an oligomeric compound and a deprotection reagent in solution in a flow filtration system.

As used herein, “depurination” means a hydrolytic cleavage of adenine or guanine from a nucleoside to leave an —OH group. A depurination nucleoside is a nucleoside in which a nucleobase is replaced with an —OH group.

As used herein, “carbohydrate” means a monosaccharide, disaccharide, trisaccharide, tetrasaccharide, oligosaccharide, polysaccharide, or derivatives thereof. In certain embodiments, a carbohydrate is N-acetylgalactosamine.

As used herein, “GalNAc” means an N-acetyl galactosamine moiety, represented by the structure:

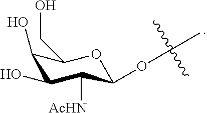

As used herein, “2′-deoxynucleoside” means a nucleoside according to the structure:

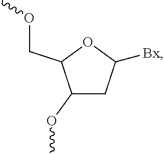

wherein Bx is a nucleobase.

As used herein, “2′-deoxy sugar moiety” means the sugar moiety of a 2′-deoxynucleoside. As indicated in the above structure, a 2′-deoxy sugar moiety can have any stereochemistry. For example, 2′-deoxy sugar moieties include, but are not limited to 2′-β-D-deoxyribosyl sugar moieties and 2′-β-D-deoxyxylosyl sugar moieties.

As used herein, “2′-β-D-deoxyribosyl nucleoside” means a nucleoside according to the structure:

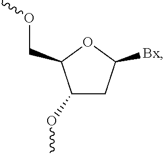

wherein Bx is a nucleobase.

As used herein, “2′-β-D-deoxyribosyl sugar moiety” means the sugar moiety of a 2′-β-D-deoxyribosyl nucleoside. The nucleobase of a 2′-deoxynucleoside or 2′-13-D-deoxyribosyl nucleoside may be a modified nucleobase or any natural nucleobase, including but not limited to an RNA nucleobase (uracil).

As used herein, “ribo-2′-MOE nucleoside” means a nucleoside according to the structure:

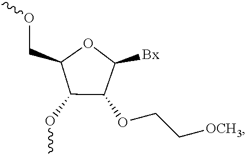

wherein Bx is a nucleobase.

As used herein, “ribo-2′-MOE sugar moiety” means the sugar moiety of a 2′-MOE nucleoside as defined herein.

As used herein, “MOE” means an —OCH₂CH₂OCH₃ group.

As used herein, “2′-OMe nucleoside” means a nucleoside according to the structure:

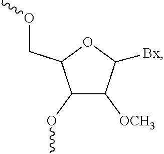

wherein Bx is a nucleobase.

As used herein, “2′-OMe sugar moiety” means the sugar moiety of a 2′-OMe nucleoside. As indicated in the above structure, a 2′-OMe sugar moiety can have any stereochemistry. For example, 2′-OMe sugar moieties include, but are not limited to 2′-OCH₃-β-D-xylosyl sugar moieties, 2′-OCH₃-α-L-ribosyl sugar moieties, and ribo-2′-OMe sugar moieties as defined herein.

As used herein, “Ribo-2′-OMe nucleoside” means a nucleoside according to the structure:

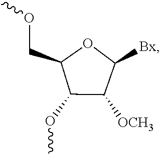

wherein Bx is a nucleobase.

As used herein, “ribo-2′-OMe sugar moiety” means the sugar moiety of a ribo-2′-OMe nucleoside.

As used herein, “2′-F nucleoside” means a nucleoside according to the structure:

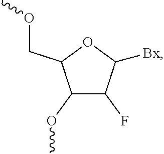

wherein Bx is a nucleobase.

As used herein, “2′-F sugar moiety” means the sugar moiety of a 2′-F nucleoside. As indicated in the above structure, a 2′-F sugar moiety can have any stereochemistry. For example, 2′-F sugar moieties include, but are not limited to, 2′-F-β-D-xylosyl sugar moieties, 2′-F-β-D-arabinosyl sugar moieties, 2′-F-α-L-ribosyl sugar moieties, and ribo-2′-F sugar moieties as defined herein.

As used herein, “ribo-2′-F nucleoside” means a nucleoside according to the structure:

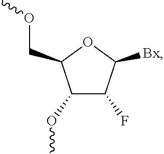

wherein Bx is a nucleobase.

As used herein, “ribo-2′-F sugar moiety” means the sugar moiety of a ribo-2′-F nucleoside as defined herein.

As used herein, “2′-NMA nucleoside” means a nucleoside according to the structure:

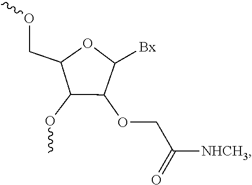

wherein Bx is a nucleobase.

As used herein, “2′-NMA sugar moiety” means the sugar moiety of a 2′-NMA nucleoside.

As used herein, “ribo-2′-NMA nucleoside” means a nucleoside according to the structure:

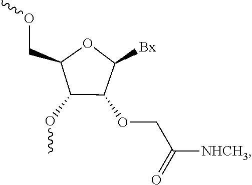

wherein Bx is a nucleobase.

As used herein, “ribo-2′-NMA sugar moiety” means the sugar moiety of a ribo-2′-NMA nucleoside.

As used herein, “2′-substituted” in reference to a sugar moiety means a furanosyl sugar moiety comprising at least one 2′-substituent group other than H or OH. As used herein, “2′-substituted nucleoside” means a nucleoside comprising a 2′-substituted furanosyl sugar moiety.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “bicyclic sugar” or “bicyclic sugar moiety” means a modified sugar moiety comprising two rings, wherein the second ring is formed via a bridge connecting two of the atoms in the first ring thereby forming a bicyclic structure, wherein the first ring of the bicyclic sugar moiety is a furanosyl ring. Examples of bicyclic sugar moieties include LNA (locked nucleic acid) sugar moiety and cEt sugar moiety as defined herein. A “bicyclic nucleoside” is a nucleoside comprising a bicyclic sugar moiety.

As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage.

As used herein, “cleavable moiety” means a bond or group of atoms that is cleaved under physiological conditions, for example, inside a cell, an animal, or a human.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a covalently bound group of atoms that modifies one or more pharmacological properties of a molecule compared to the identical molecule lacking the conjugate moiety, including but not limited to pharmacodynamics, pharmacokinetics, stability, binding, absorption, tissue distribution, cellular distribution, cellular uptake, charge, and clearance.

As used herein, “constrained ethyl nucleoside” or “cEt nucleoside” means

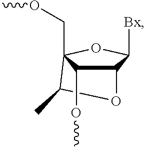

wherein Bx is a nucleobase.

“Constrained ethyl” or “cEt” or “cEt sugar moiety” means the sugar moiety of a cEt nucleoside.

As used herein, “deoxy region” means a region of 5-12 contiguous nucleotides, wherein at least 70% of the nucleosides comprise a 2′-deoxy sugar moiety. In certain embodiments, a deoxy region is the gap of a gapmer.

As used herein, “internucleoside linkage” is the covalent linkage between adjacent nucleosides in an oligonucleotide. As used herein “modified internucleoside linkage” means any internucleoside linkage other than a phosphodiester internucleoside linkage.

As used herein, “linker” means a group of atoms configured to link a conjugate moiety to an oligonucleotide.

As used herein, “linked nucleosides” are nucleosides that are connected in a contiguous sequence (i.e., no additional nucleosides are presented between those that are linked).

As used herein, “motif” means the pattern of unmodified and/or modified sugar moieties, nucleobases, and/or internucleoside linkages, in an oligonucleotide.

As used herein, “modified nucleoside” means a nucleoside comprising a modified nucleobase and/or a modified sugar moiety.

As used herein, “modified sugar moiety” means a sugar moiety of a nucleoside other than 2′-β-D-deoxyribosyl sugar moiety (the sugar moiety of unmodified DNA) or f D-ribosyl sugar moiety (the sugar moiety of unmodified RNA). Modified sugar moieties include, but are not limited to, stereo-non-standard sugar moieties, substituted sugar moieties, bicyclic sugar moieties, abasic sugar moieties, and sugar surrogates.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is an example of a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide comprising one or more modified nucleosides or having one or more modified internucleoside linkages. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “stabilized phosphate group” means a 5′-phosphate analog that is metabolically more stable than a 5′-phosphate as naturally occurs on DNA or RNA.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of a stereorandom phosphorothioate or mesyl phosphoramidate internucleoside linkage.

As used herein, “stereo-standard nucleoside” means a nucleoside comprising a non-bicyclic β-D-ribosyl sugar moiety.

As used herein, “stereo-non-standard nucleoside” means a nucleoside comprising a non-bicyclic furanosyl sugar moiety having a configuration other than that of a stereo-standard sugar moiety.

As used herein, “sugar moiety” means any sugar moiety described herein and may be an unmodified sugar moiety or a modified sugar moiety. As used herein, “unmodified sugar moiety” means a β-D-ribosyl moiety, as found in natural RNA (an “unmodified RNA sugar moiety”), or a 2′-β-D-deoxyribosyl sugar moiety, as found in natural DNA (an “unmodified DNA sugar moiety”). As used herein, “modified sugar moiety” or “modified sugar” means a modified furanosyl sugar moiety or a sugar surrogate.

As used herein, “sugar surrogate” means a moiety that can link a nucleobase to another group, such as an internucleoside linkage, conjugate group, or terminal group in an oligonucleotide, but which is not a furanosyl sugar moiety or a bicyclic sugar moiety. Modified nucleosides comprising sugar surrogates can be incorporated into one or more positions within an oligonucleotide and such oligonucleotides are capable of hybridizing to complementary oligomeric compounds or target nucleic acids. Examples of sugar surrogates include GNA (glycol nucleic acid), FHNA (fluoro hexitol nucleic acid), morpholino, and other structures described herein and known in the art.

As used herein, “terminal group” means a chemical group or group of atoms that is covalently linked to a terminus of an oligonucleotide.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-deoxynucleoside. In certain embodiments, the gap comprises one 2′-substituted nucleoside at position 1, 2, 3, 4, or 5 of the gap, and the remainder of the nucleosides of the gap are 2′-deoxynucleosides. As used herein, the term “MOE gapmer” indicates a gapmer having a gap comprising 2′-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “cell-targeting moiety” means a conjugate group or portion of a conjugate group that is capable of binding to a particular cell type or particular cell types.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

### CERTAIN EMBODIMENTS

Embodiment 1. A method for preparing an oligomeric compound comprising a modified oligonucleotide, comprising:
   a) providing a flow filtration system comprising a protected oligomeric compound solution carrying a protected oligomeric compound, wherein the protected oligomeric compound comprises a protected functional group;
   b) adding a deprotection reagent to the protected oligomeric compound solution, creating a product solution comprising the deprotected oligomeric compound and a deprotection byproduct, wherein the deprotection byproduct is soluble in the product solution; and
   c) circulating the product solution across the semi-permeable membrane for a period sufficient to separate substantially all of the deprotection byproduct from the deprotected oligomeric compound.
Embodiment 2. The method of Embodiment 1, wherein the protected functional group is attached at the 5′-terminal hydroxyl group of the modified oligonucleotide.
Embodiment 3. The method of any one of the preceding Embodiments, wherein the protected functional group is an amine or a hydroxyl.
Embodiment 4. The method of any one of the preceding Embodiments, wherein the protected functional group is a primary amine.
Embodiment 5. The method of any preceding Embodiment, wherein the protected functional group comprises a trityl group.
Embodiment 6. The method of any one of the preceding Embodiments, wherein the trityl group comprises a methoxy.
Embodiment 7. The method of any one of the preceding Embodiments, wherein the trityl group is a 4-monomethoxytrityl or 4-,4′-dimethoxytrityl group.
Embodiment 8. The method of any one of the preceding Embodiments, wherein the trityl group is a 4-monomethoxy trityl (MMT) group.
Embodiment 9. The method of any one of the preceding Embodiments, wherein the oligomeric compound comprises a linker that links the modified oligonucleotide with the protected functional group.
Embodiment 10. The method of Embodiment 9, wherein the linker is an alkyl.
Embodiment 11. The method of Embodiment 10, wherein the alkyl is n-hexyl.
Embodiment 12. The method of any one of the preceding Embodiments, wherein the protected oligomeric compound solution dissolves the protected oligomeric compound.
Embodiment 13. The method of any one of the preceding Embodiments, wherein the deprotection reagent comprises an acid.
Embodiment 14. The method of Embodiment 13, wherein the acid is acetic acid.
Embodiment 15. The method of Embodiment 13, wherein the acid is glacial acetic acid added in 1-2% w/w relative to the volume of the protected oligomeric compound solution.
Embodiment 16. The method of any one of the preceding Embodiments, wherein the product solution has a pH of 3-5.
Embodiment 17. The method of any one of the preceding Embodiments, further comprising heating the product solution.
Embodiment 18. The method of any one of the preceding Embodiments, wherein the product solution is maintained at a temperature of 30-60° C., or about 40° C.
Embodiment 19. The method of any one of the preceding Embodiments, wherein the flow filtration system is a diafiltration system.
Embodiment 20. The method of any one of the preceding Embodiments, wherein circulating the product solution comprises a diafiltration to remove the deprotection byproduct.
Embodiment 21. The method of any one of the preceding Embodiments, wherein diafiltration to remove the deprotection byproduct comprises circulating the product solution against 3 to 20 diavolumes of a diafiltration solution.
Embodiment 22. The method of any one of the preceding Embodiments, wherein the diafiltration solution comprises a salt.
Embodiment 23. The method of any one of the preceding Embodiments, wherein the salt is sodium acetate.
Embodiment 24. The method of any one of the preceding Embodiments, wherein the diafiltration solution comprises an alcohol.
Embodiment 25. The method of Embodiment 24, wherein the alcohol is methanol.
Embodiment 26. The method of any one of the preceding Embodiments, wherein the diafiltration solution comprises 30-90% methanol and 0.01-0.5 M sodium acetate.
Embodiment 27. The method of any one of the preceding Embodiments, wherein the semi-permeable membrane is characterized by a molecular weight cutoff of 1-5 kDa, or about 2 kDa.
Embodiment 28. The method of any one of the preceding Embodiments, further comprising a concentration step in which the protected oligomeric compound solution is partially removed, whereby the concentration of the protected oligomeric compound in the protected oligomeric compound solution is increased.
Embodiment 29. The method of any one of the preceding Embodiments, wherein the concentration step comprises ultrafiltration via the semi-permeable membrane.
Embodiment 30. The method of any one of the preceding Embodiments, wherein the semi-permeable membrane is a cellulose membrane.
Embodiment 31. The method of any one of the preceding Embodiments, wherein recovery of the deprotected oligomeric compound is at least about 85%, 90%, or 95% relative to the amount of protected oligomeric compound.
Embodiment 32. The method of any one of the preceding Embodiments, wherein depurination of the deprotected oligomeric compound is less than about 1% or 0.5%.
Embodiment 33. The method of any one of the preceding Embodiments, wherein at least about 1 kg of deprotected oligomeric compound is recovered.
Embodiment 34. The method of any one of the preceding Embodiments, wherein the modified oligonucleotide consists of 10-30 linked nucleosides, for example 16-23, 16, or 20 linked nucleosides.
Embodiment 35. The method of any one of the preceding Embodiments, wherein the modified oligonucleotide comprises adenine, cytosine, 5-methylcytosine, guanine, thymine, and/or uracil nucleobases.
Embodiment 36. The method of any one of the preceding Embodiments, wherein the modified oligonucleotide comprises a nucleoside sugar moiety selected from the group consisting of 2′-deoxyribosyl sugar moiety, 2′-MOE sugar moiety, LNA sugar moiety, cEt sugar moiety, 2′-NMA sugar moiety, 2′-F sugar moiety, and 2′-OMe sugar moiety, optionally wherein the modified oligonucleotide consists of nucleosides comprising sugar moieties selected from 2′-deoxyribosyl sugar moiety, 2′-MOE sugar moiety, LNA sugar moiety, cEt sugar moiety, 2′-NMA sugar moiety, 2′-F sugar moiety, and 2′-OMe sugar moiety.
Embodiment 37. The method of any one of the preceding Embodiments, wherein the modified oligonucleotide has a gapmer sugar motif.
Embodiment 38. The method of any one of the preceding Embodiments, wherein the modified oligonucleotide comprises a central region of 7-12 nucleosides flanked on the 5′-side by a 5′-external region consisting of 1-6 linked 5′-region nucleosides and on the 3′-side by a 3′-external region consisting of 1-6 linked 3′-region nucleosides; wherein each of the 5′-region nucleosides is a modified nucleoside, and each of the 3′-region nucleosides is a modified nucleoside.
Embodiment 39. The method of any one of the preceding Embodiments, wherein the modified oligonucleotide comprises a central region of 10 nucleosides flanked on the 5′-side by a 5′-external region consisting of 5 linked 5′-region nucleosides and on the 3′-side by a 3′-external region consisting of 5 linked 3′-region nucleosides; wherein each of the 5′-region nucleosides is a modified nucleoside, and each of the 3′-region nucleosides is a modified nucleoside.
Embodiment 40. The method of any one of the preceding Embodiments, wherein the central region comprises linked 2′-β-D-deoxyribosyl nucleosides, each 3′-region nucleoside is selected from a ribo-2′-MOE nucleoside and a cEt nucleoside, and each 5′-region nucleoside is selected from a ribo-2′-MOE nucleoside and a cEt nucleoside.
Embodiment 41. The method of any one of the preceding Embodiments, wherein the modified oligonucleotide comprises or consists of nucleosides comprising sugar moieties selected from 2′-deoxyribosyl sugar moieties, 2′-OMe sugar moieties, and 2′-F sugar moieties.
Embodiment 42. The method of any one of the preceding Embodiments, wherein each nucleoside in the modified oligonucleotide comprises a modified sugar moiety.
Embodiment 43. The method of any one of the preceding Embodiments, wherein the modified oligonucleotide comprises a modified internucleoside linkage.
Embodiment 44. The method of any one of the preceding Embodiments, wherein the modified oligonucleotide comprises a modified internucleoside linkage selected from a phosphorothioate internucleoside linkage and a phosphoramidate internucleoside linkage, optionally wherein the modified oligonucleotide includes only internucleoside linkages selected from phosphorothioate internucleoside linkages, mesyl phosphoramidate internucleoside linkages, and phosphodiester internucleoside linkages.
Embodiment 45. The method of any one of the preceding Embodiments, wherein the modified oligonucleotide comprises a phosphorothioate internucleoside linkage.
Embodiment 46. The method of any one of the preceding Embodiments, wherein the protected oligomeric compound consists of a modified oligonucleotide, a linker, and a protected functional group.
Embodiment 47. The method of any one of the preceding Embodiments, wherein the oligomeric compound comprises a conjugate group or a stabilized phosphate group.
Embodiment 48. The method of any one of the preceding Embodiments, wherein the conjugate group comprises at least one GalNAc moiety, and optionally a triantennary GalNAc cell-targeting moiety.
Embodiment 49. The method of any one of the preceding Embodiments, wherein the conjugate group has the structure:

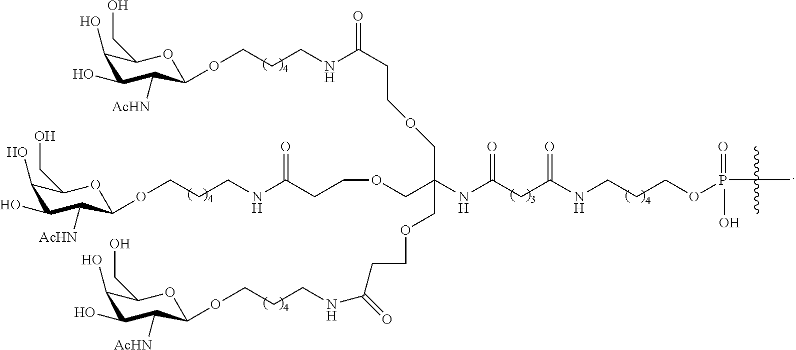

Embodiment 50. An oligomeric compound prepared by the method of any one of the preceding Embodiments.

### Certain Processes for Synthesis of Oligonucleotides

Solid-phase synthesis of oligomeric compounds using phosphoramidite intermediates is performed by an iterative process wherein a series of chemical reactions are performed to assemble the desired oligonucleotide on a solid support. After synthesis the support-bound, protected oligomeric compound is cleaved from the solid support. Typically, the 5′-terminal protecting group may be a 4,4′-dimethoxytrityl (DMT) or 4-methoxytrityl (monomethoxytrityl or MMT) group. The protected oligomeric compound then undergoes purification via column chromatography. After purification, the protecting group is cleaved in a final deprotection step. When the protecting group is a trityl, there are two common procedures for purification and final detritylation step: strong anion exchange chromatography with on-column detritylation (SAX-OCD) and reversed-phase high performance liquid chromatography (RP-HPLC) followed by isolation via ethanol precipitation and then solution-phase detritylation.

In SAX-OCD the protected oligomeric compound is bound to a chromatography column packed with a resin typically functionalized with a quaternary amine. The purification column containing the resin-bound oligomeric compound is then charged with a deprotection medium (where the protecting group is a trityl, typically the deprotection medium may be acetic acid, e.g., 80% acetic acid) to cleave the protecting group from the remainder of the oligomeric compound. After a prescribed reaction time at a certain temperature the oligomeric compound is deprotected. The acid solution and trityl alcohol formed from the detritylation reaction are eluted from the column, leaving behind the deprotected oligomeric compound bound to the column. The oligomeric compound is then eluted from the column with a mobile phase containing a certain concentration of base (such as sodium hydroxide) and salt (such as sodium chloride). The resulting eluate contains the deprotected oligomeric compound in an aqueous, basic buffer that contains salt. The eluate is typically concentrated, neutralized, and desalted using tangential flow filtration (TFF).

The protected oligomeric compound may otherwise be first purified by RP-HPLC. During RP-HPLC, the protecting group serves as a chromatographic handle that aids in separation of the desired oligonucleotide product from impurities. The protected oligomeric compound may be eluted with an aqueous mobile phase containing a polar solvent (such as methanol) and salt (such as sodium acetate). The resulting eluate is then isolated from the mobile phase via ethanol precipitation followed by reconstitution with purified water. The aqueous solution of protected oligomeric compound then undergoes solution-phase deprotection, in which acid (where the protecting group is trityl, glacial acetic acid may be used) is introduced to cleave the protecting group from the oligomeric compound. The deprotected oligomeric compound is precipitated in ethanol and reconstituted with purified water two more times to isolate the oligomeric compound from the byproduct formed during the deprotection reaction.

Tangential Flow Filtration Tangential flow filtration (TFF), also known as cross flow filtration, is a processing technique wherein the feed flow travels tangentially across the surface of the filter surface; this differs from traditional dead-end filtration in which the feed flow is directed perpendicular to the filter surface. TFF has an advantage over dead-end filtration in that the filter cake (species too big to pass through the filter membrane) is continuously washed away during operation, which helps to prevent fouling (clogging) of the membrane. TFF filter membranes are rated with nominal molecular weight cutoffs (MWCOs), for which molecules smaller than the MWCO can pass through the membrane and molecules larger than the MWCO cannot pass through the membrane. In the context of oligonucleotides, the membrane MWCO is chosen to be smaller than the oligonucleotide molecular weight so the oligomeric compound is retained while solvents, salts, and small molecule impurities pass through into the permeate stream. In certain embodiments, the membrane is a cellulose membrane.

There are two modes of TFF operation: ultrafiltration and diafiltration. Ultrafiltration is typically used as a concentration step. During ultrafiltration, the product solution is fed into the process tank (retentate tank) at the same rate that the permeate exits the membrane, thereby maintaining a constant volume in the system with an ever-increasing oligonucleotide concentration. Diafiltration is typically used as a buffer exchange step. During diafiltration, a buffer solution (such as purified water) is fed into the process tank (retentate tank) at the same rate that the permeate exits the membrane, thereby keeping the oligonucleotide concentration constant while replacing the oligonucleotide solvent with the new buffer. For example, an oligonucleotide solution containing a high concentration of salt can be desalted by diafiltration if purified water is used as the feed buffer.

TFF is typically used in the manufacture of oligonucleotides for concentrating and desalting product that has been purified by strong anion exchange (SAX) chromatography. The SAX eluates collected from this purification technique are often dilute (oligonucleotide concentration of about 2-5 mg/mL) and contain sodium chloride (salt concentration of about 0.5-1.0 M). The dilute SAX eluates are typically concentrated to approximately 50 mg/mL to reduce the working volume of solution, then are diafiltered against purified water to remove sodium chloride.

In certain embodiments, the instant methods may comprise a step in which a protected oligomeric compound is deprotected in a flow filtration system, for example, a tangential flow filtration system, or a vessel thereof. The deprotection reaction may be conducted via the addition of an acid (such as glacial acetic acid) to the mixing oligonucleotide solution and controlling the reaction temperature for a set amount of time. The optimized deprotection conditions will vary based on a variety of factors: oligonucleotide concentration, oligonucleotide sequence, protecting group (e.g., dimethoxy trityl (DMT) versus monomethoxytrityl (MMT)). For example, in certain embodiments, MMT-protected oligonucleotides are detritylated by adding 1.5% (w/w) glacial acetic acid with respect to concentrated oligonucleotide solution, warming to 40° C., and mixing for 6 hours. Once the oligonucleotide has been fully deprotected, the reaction is quenched by adjusting the pH and/or the temperature. For example, removal of DMT-protecting groups is typically performed at pH 3.5 and 22° C., so the reaction would be quenched by adding a base (such as sodium hydroxide) to neutralize the pH. Removal of MMT-protecting groups is typically performed at pH 4.5 and 40° C., so the reaction would be quenched by cooling the solution back to 22° C. The resulting solution contains the deprotected oligonucleotide, dissolved trityl alcohol, salt, and buffer.

In a step, the trityl alcohol byproduct may be removed by diafiltration against a buffer containing an alcohol, e.g., methanol (“diafiltration solution”). The diafiltration solution may comprise or consist of 30-90% methanol and 0.01-0.5 M sodium acetate, or 50-80% v/v methanol and 0.05-0.20 M sodium acetate, in order to keep both the oligonucleotide and trityl alcohol dissolved. When diafiltering on a membrane with MWCO of 1-5 kDa, the oligonucleotide (6-9 kDa) is too large to pass through the membrane while trityl alcohol (0.3 kDa) is small enough to pass through into the permeate. Diafiltration of the oligonucleotide solution against 3 or more diavolumes of the diafiltration solution may provide >99% rejection of trityl alcohol. The resulting solution contains the deprotected oligonucleotide, sodium acetate, and methanol.

In a step, an optional buffer exchange is performed in order to isolate the deprotected oligonucleotide in the buffer of choice for further downstream processing. For example, the oligonucleotide solution can be diafiltered with purified water (thereby removing methanol and salt) and then isolated via freeze drying. Alternatively, the oligonucleotide solution can be diafiltered with salt or salt buffer in preparation for GalNAc-conjugation. In certain embodiments, the deprotected oligonucleotide is diafiltered against an aqueous solution (a wash solution) to remove an organic solvent such as methanol. The wash solution may comprise a salt, for example, sodium acetate. The wash solution may be free of organic solvent.

The methods described herein may be implemented in discovery, preparatory, or process syntheses. For example, in certain embodiments, more than 1 kg of the deprotected oligomeric compound may be synthesized (e.g., in a single batch) by a method described herein.

In certain embodiments, the methods described herein are useful for purifying mixtures containing oligomeric compounds comprising oligonucleotides consisting of linked nucleosides. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase) and/or at least one modified internucleoside linkage). The present disclosure provides processes of preparing oligomeric compounds comprising modified oligonucleotides that have any number or combinations of modifications described herein.

In the industry standard process for solution phase trityl-protected oligonucleotide deprotection, the detritylation reaction is performed under aqueous conditions. Trityl alcohol is not soluble in water, so a precipitate can be produced which may clog transfer lines. In some embodiments of the instant process, byproduct trityl alcohol may remain in solution. In certain embodiments, the instant process provides material of a higher purity compared to a SAX-OCD process commonly used. SAX-OCD involves extended contact times with acid during the detritylation reaction which can lead to greater depurination. In SAX-OCD, the oligonucleotide product is eluted with a high pH buffer, which can then cleave depurinated nucleosides resulting in a class of impurities called early eluting impurities. The instant processes may limit the formation of potential degradation products associated with the pH conditions during SAX-OCD.

In certain embodiments, the instant processes may also reduce manufacturing times compared to existing processes. There are several factors that can impact the manufacturing time such as batch size, equipment size/capacity, and working hours (single shift versus 24-hour operation). For example, in certain embodiments, the instant process may reduce from 23-35 hours processing time using a typical process to 14 hours.

In certain embodiments, the instant process may also use less organic solvent than a commonly used process. For example, the volume of alcohol utilized by the instant process may be less than the volume of ethanol utilized in a commonly used process.

### I. Certain Oligomeric Compounds

In certain embodiments, provided herein are oligomeric compounds comprising oligonucleotides, which consist of linked nucleosides, wherein the oligomeric compound is prepared by a method described herein. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase, or both a modified sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non-bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃(“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃. In certain embodiments, a modified oligonucleotide comprises one or more of a 2′-MOE nucleoside, a 2′-OMe nucleoside, a 2′-F nucleoside, and a 2′-NMA nucleoside. In certain embodiments, the modified oligonucleotide comprises a stereo-non-standard sugar moiety.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring f-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently selected from: H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), and sulfoxyl (S(═O)-J₁); and each J₁ and J₂ is, independently selected from: H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, and a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

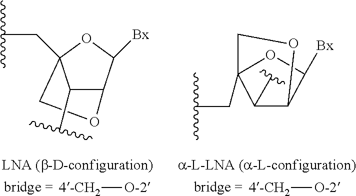

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, C J. Bioorg. & Med. Chem. 2002, 10, 841-854), fluoro HNA:

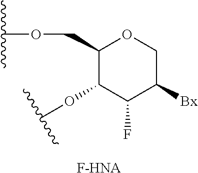

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

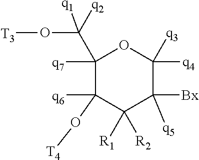

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

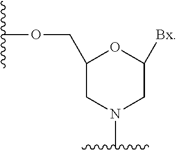

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

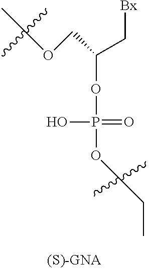

where Bx represents any nucleobase.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15. Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“—O—P(═O)(OH)”) (also referred to as unmodified or naturally occurring linkages), phosphotriesters, methylphosphonates, phosphoramidates, and phosphorothioates (“—O—P(═S)(OH)—”), and phosphorodithioates (“—O—P(═S)(SH)”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO2021/030778, incorporated by reference herein. In certain embodiments, a modified internucleoside linkage comprises the formula:

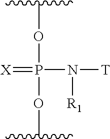

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₂-C₆ alkenyl, C₂-C₆ alkynyl, substituted C₁-C₆ alkyl, substituted C₂-C₆ alkenyl substituted C₂-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

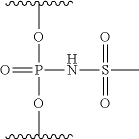 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

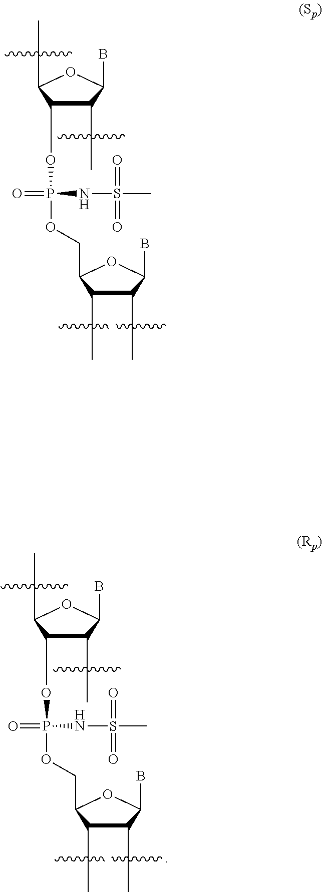

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate linkage or mesyl phosphoramidate. Nonetheless, as is well understood by those of skill in the art, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

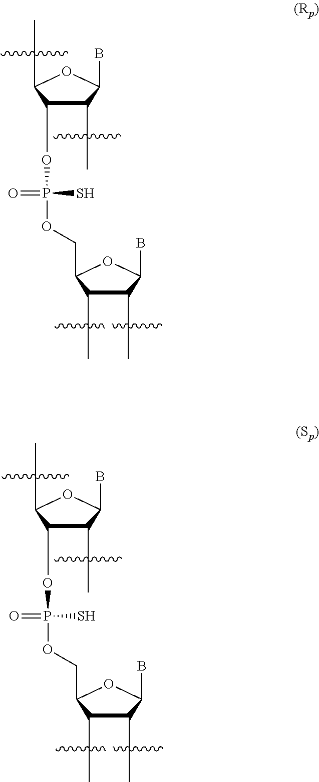

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

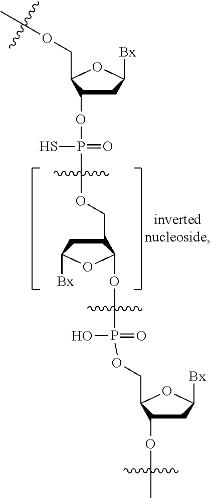

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

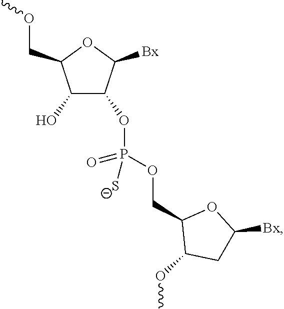

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligomeric compounds or oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Uniformly Modified Oligonucleotides

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified nucleotide comprises the same 2′-modification.

### Gapmer Oligonucleotides

In certain embodiments, modified oligonucleotides comprise or consist of a sequence of nucleosides having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap region (the 3′-most nucleoside of the 5′-wing and the 5′-most nucleoside of the 3′-wing) differ from the sugar moiety of the neighboring gap nucleosides, thus defining the boundary between the wings and the gap region (i.e., the wing/gap junction). In certain embodiments, the sugar moieties within the gap are the same as one another. In certain embodiments, the gap region includes one or more nucleoside having a sugar moiety that differs from the sugar moiety of one or more other nucleosides of the gap. In certain embodiments, the second nucleoside from the 5′-most gap nucleoside comprises a 2′-OMe sugar moiety, and all other gap nucleosides comprise 2′-deoxy sugar moieties. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing region of a gapmer is a modified nucleoside. In certain embodiments, at least one nucleoside of each wing region of a gapmer is a modified nucleoside. In certain embodiments, at least two nucleosides of each wing region of a gapmer are modified nucleosides. In certain embodiments, at least three nucleosides of each wing region of a gapmer are modified nucleosides. In certain embodiments, at least four nucleosides of each wing region of a gapmer are modified nucleosides.

In certain embodiments, the gap region of a gapmer comprises 7-12 nucleosides. In certain embodiments, each nucleoside of the gap region of a gapmer is a 2′-deoxynucleoside. In certain embodiments, at least one nucleoside of the gap region of a gapmer is a modified nucleoside.

In certain embodiments, the gapmer is a deoxy gapmer, i.e., a gapmer that comprises a deoxy region. In certain embodiments, the nucleosides on the gap side of each wing/gap junction are unmodified 2′-deoxynucleosides and the nucleosides on the wing sides of each wing/gap junction are modified nucleosides. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxy sugar moiety. In certain embodiments, at least one, or exactly one, nucleoside of the gap of a gapmer comprises a 2′-OMe sugar moiety.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing region and the gap region nucleosides comprise 2′-deoxy sugar moieties. Thus, a 3-10-3 cEt gapmer consists of 3 linked cEt nucleosides in the 5′-wing, 10 linked 2′-deoxynucleosides in the gap, and 3 linked cEt nucleosides in the 3′-wing. Similarly, a 2-12-2 cEt gapmer consists of 2 linked cEt nucleosides in the 5′-wing, 12 linked 2′-deoxynucleosides in the gap, and 2 linked cEt nucleosides in the 3′-wing. A 5-10-5 MOE gapmer consists of 5 linked ribo-2′-MOE nucleosides in the 5′-wing, 10 linked 2′-deoxynucleosides in the gap, and 5 linked ribo-2′-MOE nucleosides in the 3′-wing.

In certain embodiments, a modified oligonucleotides is a 5-10-5 MOE gapmer. In certain embodiments, a modified oligonucleotide is a 3-10-3 cEt gapmer. In certain embodiments, a modified oligonucleotide is a 3-10-4 MOE gapmer.

### Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the gap region of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynylpyrimidine.

### 2. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (“—O—P(═O)(OH)—”). In certain embodiments, each internucleoside linking group of a modified oligonucleotide is a phosphorothioate internucleoside linkage (“—O—P(═O)(SH)—”). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

In certain embodiments, the sugar motif of a modified oligonucleotide is a gapmer and the internucleoside linkages within the gap region are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap region comprises at least one Sp, Sp, Rp motif. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein is an oligomeric compound consisting of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near, e.g., one or two nucleobases from, the 3′-end of oligonucleotides. In certain embodiments, conjugate groups (or terminal groups) are attached at the 5′-end of oligonucleotides. In certain embodiments, conjugate groups are attached near, e.g., one or two nucleobases from, the 5′-end of oligonucleotides. In certain embodiments, conjugate groups (or terminal groups) are attached at the 3′-terminal nucleoside of an oligonucleotide. In certain embodiments, conjugate groups (or terminal groups) are attached at the 5′-terminal nucleoside of an oligonucleotide.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, oligomeric compounds comprise a conjugate group comprising a cell-targeting moiety having an affinity for transferrin receptor (TfR) (also known as TfR1 or CD71). In certain embodiments, the conjugate group comprises an anti-TfR1 antibody or fragment thereof. In certain embodiments, the anti-TfR1 antibody or fragment thereof can be any known in the art including but not limited to those described in WO/1991/004753; WO/2013/103800; WO/2014/144060; WO/2016/081643; WO2016/179257; WO/2016/207240; WO/2017/221883; WO/2018/129384; WO/2018/124121; WO/2019/151539; WO/2020/132584; WO/2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv. In certain embodiments, the conjugate group comprises a protein or peptide capable of binding TfR1. In certain embodiments, the protein or peptide capable of binding TfR1 can be any known in the art including but not limited to those described in WO/2019/140050; WO/2020/037150; WO/2020/124032; and U.S. Pat. No. 10,138,483. In certain embodiments, the conjugate group comprises an aptamer capable of binding TfR1. In certain embodiments, the aptamer capable of binding TfR1 can be any known in the art including but not limited to those described in WO/2013/163303; WO/2019/033051; and WO/2020/245198.

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, the conjugate group has the following structure:

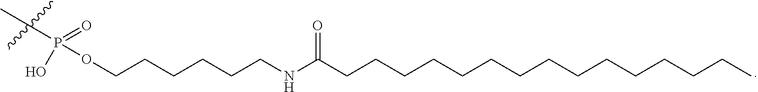 [SMILES: *P(=O)(O)OCCCCCCNC(=O)CCCCCCCCCCCCCCC]

In certain embodiments, an oligomeric compound comprises a 6-palmitamidohexyl phosphate conjugate group attached to the 5′-OH of a modified oligonucleotide wherein the structure for the conjugate group is:

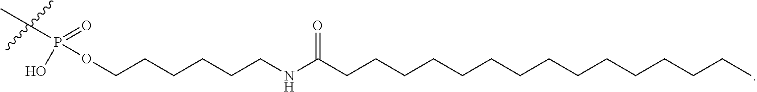 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Linkers

In some embodiments, an oligomeric compound may comprise a linker or a conjugate linker. In some embodiments, an oligomeric compound may consist of a modified oligonucleotide, a linker, and optionally a protected functional group. In such embodiments, the linker may be a terminal moiety.

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of linkers or conjugate linkers include but are not limited to pyrrolidine, 8-amino-3,6-dioxaoctanoic acid (ADO), succinimidyl 4-(N-maleimidomethyl) cyclohexane-1-carboxylate (SMCC), 6-aminohexanol (THA), and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine, or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Certain Oligomeric Duplexes

Certain embodiments are directed to oligomeric duplexes comprising a first oligomeric compound and a second oligomeric compound, wherein at least one of the first oligomeric compound and the second oligomeric compound is prepared by a method described herein.

Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

In certain embodiments, an oligomeric duplex comprises: a first oligomeric compound comprising a first modified oligonucleotide a second oligomeric compound comprising a second modified oligonucleotide wherein the nucleobase sequence of the second modified oligonucleotide comprises a complementary region that is at least 90% complementary to an equal length portion of the first modified oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide.

### 3. Conjugate Groups

In certain embodiments, a conjugate group comprises a cell-targeting moiety. In certain embodiments, conjugate groups comprise cell-targeting moieties that have at least one tethered ligand. In certain embodiments, cell-targeting moieties comprise two tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactosamine (GalNAc), mannose, glucose, glucosamine and fucose. In certain embodiments, each ligand is N-acetyl galactosamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, compounds comprise a conjugate group having the formula:

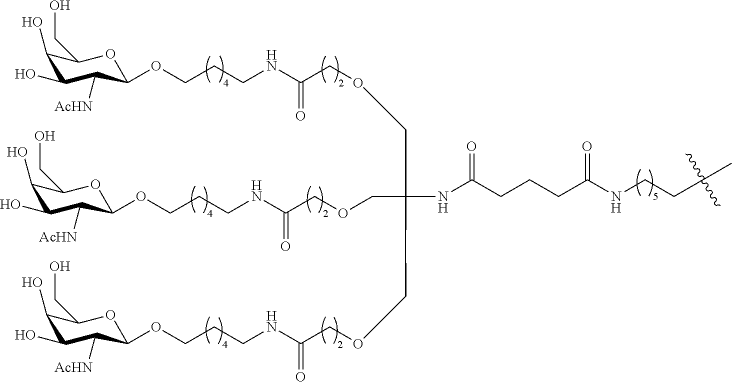

Representative United States patents, United States patent application publications, international patent application publications, and other publications that teach the preparation of certain of the above noted conjugate groups, compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011,19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron. 1997, 53, 759-770.

In certain embodiments, modified oligonucleotides comprise a gapmer or uniformly modified sugar motif and a conjugate group comprising at least one, two, or three GalNAc ligands. In certain embodiments, compounds comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Komilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, oligomeric compounds comprise a conjugate group comprising a cell-targeting moiety having an affinity for transferrin receptor (TfR), also known as TfR1 and CD71. In certain embodiments, the conjugate group comprises an anti-TfR1 antibody or fragment thereof. In certain embodiments, the anti-TfR1 antibody or fragment thereof can be any known in the art including but not limited to those described in WO/1991/004753; WO/2013/103800; WO/2014/144060; WO/2016/081643; WO2016/179257; WO/2016/207240; WO/2017/221883; WO/2018/129384; WO/2018/124121; WO/2019/151539; WO/2020/132584; WO/2020/028864; U.S. Pat. Nos. 7,208,174; 9,034,329; and 10,550,188. In certain embodiments, a fragment of an anti-TfR1 antibody is F(ab′)2, Fab, Fab′, Fv, or scFv. In certain embodiments, the protein or peptide capable of binding TfR1 can be any known in the art including but not limited to those described in WO/2019/140050; WO/2020/037150; WO/2020/124032; and U.S. Pat. No. 10,138,483. In certain embodiments, the conjugate group comprises an aptamer capable of binding TfR1. In certain embodiments, the aptamer capable of binding TfR1 can be any known in the art including but not limited to those described in WO/2013/163303; WO/2019/033051; and WO/2020/245198.

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

### NONLIMITING DISCLOSURE AND INCORPORATION BY REFERENCE

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although sequences may be designated as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

Also encompassed by the disclosure are equivalents of the described embodiments.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Lab Scale Detritylation and Purification of Modified Oligonucleotides Using Tangential Flow Filtration

Modified oligonucleotides intermediates were prepared, then deprotected and purified using a tangential flow filtration (TFF) skid.

Compound 1-(PO₄)—(CH₂)₆—NH₂ is an oligonucleotide intermediate 20 nucleosides in length comprising adenine, guanine, thymine, and 5-methylcytosine nucleobases and having a sugar motif of (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif of Compound 1-(PO₄)—(CH₂)₆—NH₂ is (from 5′ to 3′): soooossssssssssooss, wherein each “s” represents a phosphorothioate internucleoside linkage, and each “o” represents a phosphodiester internucleoside linkage. Each cytosine residue is a 5-methylcytosine. As indicated, the 5′-terminal hydroxyl of the Compound 1-(PO₄)—(CH₂)₆—NH₂ is connected via phosphodiester linkage to an aminohexyl phosphate linker group. Compound 1-MMT is a 4-monomethoxytrityl (MMT)-protected oligonucleotide intermediate identical to Compound 1-(PO₄)—(CH₂)₆—NH₂, except that the 5′-NH₂ of Compound 1-MMT is capped with a MMT protecting group.

Compound 2-(PO₄)—(CH₂)₆—NH₂ is a modified oligonucleotide 16 nucleosides in length comprising adenine, guanine, and thymine nucleobases and having a sugar motif of (from 5′ to 3′): kkkddddddddddkkk; wherein each “d” represents a 2′-β-D-deoxyribosyl sugar moiety, and each “k” represents a cEt sugar moiety. The internucleoside linkage motif of Compound 2-(PO₄)—(CH₂)₆—NH₂ is (from 5′ to 3′): sssssssssssssss, wherein each “s” represents a phosphorothioate internucleoside linkage. The 5′-terminal hydroxyl of the Compound 2-(PO₄)—(CH₂)₆—NH₂ is connected via phosphodiester linkage to an aminohexyl phosphate linker group. Compound 2-MMT is an MMT-protected oligonucleotide intermediate identical to Compound 2-(PO₄)—(CH₂)₆—NH₂, except that the —NH₂ on the 5′ end of Compound 2-MMT is capped with a MMT protecting group.

Oligonucleotide intermediates Compound 1-MMT and Compound 2-MMT were synthesized using standard solid-phase synthesis techniques, cleaved from the solid support, and purified by reverse-phase column chromatography. The resulting MMT-protected oligonucleotide intermediates were then detritylated and purified using the TFF process, as described herein. MMT-protected oligonucleotide intermediates were concentrated by ultrafiltration on a 2 kDa MW cut off (MWCO) cellulose membrane. The detritylation reaction was initiated by adding 1.5% (w/w) glacial acetic acid and warming the reaction mixture to 40° C. for 5-6 hours. After detritylation, the deprotected oligonucleotide solution was diafiltered against 6 diavolumes of a buffer containing 75% (v/v) methanol with 0.05 M sodium acetate in water. IP-HPLC-UV (ion pairing high performance liquid chromatography with ultraviolet detection) analysis of the samples before and after diafiltration on the 2 kDa MWCO cellulose membrane revealed that the trityl alcohol was removed.

The detritylation reaction mixtures were sampled regularly and analyzed by IP-HPLC-UV-MS (ion pairing high performance liquid chromatography with ultraviolet detection coupled with mass spectrometry) to monitor the detritylation reaction progress. The MS peaks corresponding to the MMT-protected intermediate compounds and the deprotected compounds were identified and integrated in OpenLab ChemStation version C.01.09. To determine the amount of MMT-protected oligonucleotide remaining, the area of the MS peak corresponding to the MMT-protected intermediate compound was normalized to the sum area of the MS peak of the MMT-protected intermediate compound and the deprotected compound. Results are presented in the table below as “MMT-Protected Oligonucleotide Remaining (%)”. “N.D.” indicates that the data was not determined. The detritylation reaction obeyed first order reaction kinetics, which is typical for the solution-phase detritylation reaction.

TABLE 1
Kinetics of detritylation reaction

| | Compound 1-MMT | Compound 2-MMT |
|---|---|---|
| Time (min) | Remaining (%) | Remaining (%) |
| 0 | 76.47 | 90.75 |
| 15 | 49.20 | 58.09 |
| 30 | 35.58 | 41.44 |
| 60 | 17.44 | 21.17 |
| 120 | 4.50 | 5.30 |
| 180 | 1.22 | N.D. |
| 230 | 0.49 | N.D. |
| 240 | N.D. | 0.32 |
| 300 | 0.26 | 0.12 |
| 360 | 0.09 | N.D. |

### Example 2: Large Scale Detritylation and Purification of Modified Oligonucleotides Using Tangential Flow Filtration

Oligonucleotides intermediates were prepared on large manufacturing scale, then deprotected and purified using the TFF process or the standard process.

Compound 3-(PO₄)—(CH₂)₆—NH₂ is an oligonucleotide intermediate 20 nucleosides in length comprising adenine, guanine, thymine, and 5-methylcytosine nucleobases and having a sugar motif of (from 5′ to 3′): eeeeeddddddddddeeeee; wherein each “d” represents a 2′-O-D-deoxyribosyl sugar moiety, and each “e” represents a 2′-MOE sugar moiety. The internucleoside linkage motif of Compound 3-(PO₄)—(CH₂)₆—NH₂ is (from 5′ to 3′): ssoosssssssssssooss, wherein each “s” represents a phosphorothioate internucleoside linkage, and each “o” represents a phosphodiester internucleoside linkage. Each cytosine residue is a 5-methylcytosine. The 5′-terminal hydroxyl of Compound 3-(PO₄)—(CH₂)₆—NH₂ is connected via phosphodiester linkage to an aminohexyl phosphate linker group. Compound 3-MMT is a MMT-protected oligonucleotide intermediate identical to Compound 3-(PO₄)—(CH₂)₆—NH₂, except that the —NH₂ on the 5′ end of Compound 3-MMT is capped with a MMT protecting group.

Compound 4-(PO₄)—(CH₂)₆—NH₂ is an oligonucleotide intermediate 16 nucleosides in length comprising adenine, guanine, thymine, and 5-methylcytosine nucleobases and having a sugar motif of (from 5′ to 3′): kkkddddddddddkkk; wherein each “d” represents a 2′-β-D-deoxyribosyl sugar moiety, and each “k” represents a cEt sugar moiety. The internucleoside linkage motif of Compound 4-(PO₄)—(CH₂)₆—NH₂ is (from 5′ to 3′): sssssssssssssss, wherein each “s” represents a phosphorothioate internucleoside linkage. Each cytosine residue is a 5-methylcytosine. The 5′-terminal hydroxyl of the Compound 4-(PO₄)—(CH₂)₆—NH₂ is connected via phosphodiester linkage to an aminohexyl phosphate linker group. Compound 4-MMT is an oligonucleotide intermediate identical to Compound 4-(PO₄)—(CH₂)₆—NH₂, except that the —NH₂ on the 5′ end of Compound 4-MMT is capped with a MMT protecting group.

MMT-protected modified oligonucleotide intermediates described herein above were synthesized on a reaction scale indicated in the table below using standard techniques. MMT-protected oligonucleotide intermediates were then deprotected and purified using either the standard process, or the TFF process described herein above. In the standard process, the MMT-protected oligonucleotide intermediate is first isolated by precipitation in ethanol and reconstituted in purified water. The MMT-protected oligonucleotide intermediate is then detritylated by adding 1.5% (w/w) glacial acetic acid and warming the solution to 40° C. After detritylation, the deprotected oligonucleotide is isolated from trityl alcohol by two rounds of ethanol precipitation and reconstitution in purified water to yield pure detritylated oligonucleotide.

A sample of each oligonucleotide lot was analyzed for purity by IP-HPLC-UV-MS. The MS peaks corresponding to the MMT-protected intermediate compounds and the deprotected compounds were identified and integrated in OpenLab ChemStation version C.01.09. The area of the MS peak corresponding to the MMT-protected intermediate compound was normalized to the sum area of the MS peak of the MMT-protected intermediate and the deprotected compound to determine the amount of MMT-protected oligonucleotide remaining, presented in the table below as “MMT-Protected Oligonucleotide Remaining (%)”. MMT-Protected Oligonucleotide Remaining (%) is a measure of the completion of the detritylation reaction, where a value of not more than 0.20% is a passing result. The amount of depurinated oligonucleotide resulting from an undesired acid-mediated degradation side reaction was also analyzed and summarized in the table below as “Depurination (%)”. A Depurination (%) value of not more than 1.5% is a passing result. Although not explicitly measured, the trityl alcohol impurity was visually assessed to have been removed by the new process as evidenced by a clear solution versus a cloudy solution.

TABLE 2
Recovery and purity of large scale detritylation and tangential flow filtration

| | | | | MMT-Protected | |
|---|---|---|---|---|---|
| | Reaction | | | Oligonucleotide | |
| | scale | | Recovery | Remaining | Depurination |
| Compound No. | (g) | Process | (%) | (%) | (%) |
| 1-(PO₄)—(CH₂)₆—NH₂ | 2219 | Standard | 100.4 | 0.01 | 0.42 |
| | 2780 | Standard | 94.5 | 0.00 | 0.67 |
| | 7077 | Standard | 94.9 | 0.00 | 0.25 |
| 3-(PO₄)—(CH₂)₆—NH₂ | 1386 | Standard | 98.5 | 0.02 | 0.35 |
| | 2956 | Standard | 97.3 | 0.01 | 0.27 |
| 4-(PO₄)—(CH₂)₆—NH₂ | 2740 | TFF | 97.9 | 0.01 | 0.37 |

The TFF detritylation process uses less organic solvent and thus produces less waste than the standard process. A comparison of solvent and reagent consumption of the standard precipitation method and the TFF process is summarized in the table below. In each case the TFF process consumes less solvents and reagent than the standard process. Additionally, the volume of methanol utilized by the new process is significantly less than the volume of ethanol utilized by the standard process. Furthermore, methanol is less expensive than ethanol resulting in a decrease in production costs.

TABLE 3
Comparison of solvent and reagent consumption between standard process and
TFF process for Compound 1-(PO₄)—(CH₂)₆—NH₂ and Compound 3-(PO₄)—(CH₂)₆—NH₂

| | Compound 1-(PO₄)—(CH₂)₆—NH₂ | | Compound 3-(PO₄)—(CH₂)₆—NH₂ | |
|---|---|---|---|---|
| | Standard | TFF | Standard | TFF |
| Reaction scale (g) | 2219 | 2259 | 2956 | 3026 |
| Organic solvent | 797.7 L | 267.5 L | 1090.0 L | 260.8 L |
| volume (L) | ethanol | methanol | ethanol | methanol |
| Salt | 10.5 L of 3M | 30.2 L of 3M | 5.8 L of 3M | 12.4 kg of 2M |
| | sodium acetate | sodium acetate | sodium acetate | sodium acetate |
| | solution | solution | solution | solution |
| | | 1.2 kg sodium | | 1.43 kg sodium |
| | | chloride solid | | chloride solid |
| Glacial acetic | 0.185 | 1.32 | 0.25 | 1.27 |
| acid volume (L) | | | | |
| Purified water | 77.2 | 513.8 | 93.4 | 590.7 |
| volume (L) | | | | |
| Total waste | 885.6 | 814.0 | ~1190 | ~865 |
| volume (L) | | | | |

### Example 3: Large Scale Detritylation and Purification of Modified Oligonucleotides Using Tangential Flow Filtration

Oligonucleotides intermediates were prepared on large manufacturing scale, then deprotected and purified using the TFF detritylation process.

Compound 5-(PO₄)—(CH₂)₆—NH₂ is an oligonucleotide intermediate 21 nucleosides in length comprising adenine, guanine, thymine, and cytosine nucleobases and having a sugar motif of (from 5′ to 3′): eeyyyyyyyffyyyyyyyyee; wherein each “e” represents a 2′-MOE sugar moiety, each “y” represents a 2′-Me sugar moiety, and each “f” represents a 2′-fluoro sugar moiety. The internucleoside linkage motif of Compound 5-(PO₄)—(CH₂)₆—NH₂ is (from 5′ to 3′): ssooooooosooooooooss, wherein each “s” represents a phosphorothioate internucleoside linkage, and each “o” represents a phosphodiester internucleoside linkage. The 5′-terminal hydroxyl of Compound 5-(PO₄)—(CH₂)₆—NH₂ is connected via phosphodiester linkage to an aminohexyl phosphate linker group. Compound 5-MMT is a MMT-protected oligonucleotide intermediate identical to Compound 5-(PO₄)—(CH₂)₆—NH₂, except that the —NH₂ on the 5′ end of Compound 5-MMT is capped with a MMT protecting group.

Compound 5-MMT was synthesized and purified by RP-HPLC. Purified Compound 5-MMT (3184 g) was concentrated on the TFF system to a concentration of 50 mg/mL. The solution of concentrated intermediate (63.7 L) was warmed to 40° C., and 10% acetic acid solution (9.6 L, 0.15 volumes) was added. The acidified solution was mixed via recirculation for 390 minutes before being cooled back down to 22° C. The detritylated solution was then diafiltered with 5.0 diavolumes of a buffer containing 75% (v/v) methanol, 25% (v/v) water, and 0.0625 M sodium acetate to remove MMT-OH generated during the detritylation reaction. The resulting solution of Compound 5-(PO₄)—(CH₂)₆—NH₂ was then diafiltered with 4.0 diavolumes of wash solution containing 0.05 M sodium acetate in water to remove the methanol buffer, preparing the intermediate for subsequent manufacturing.

A sample of Compound 5-(PO₄)—(CH₂)₆—NH₂ was analyzed for purity by IP-HPLC-UV-MS using OpenLab ChemStation version C.01.09, as described herein above. Purity of the lot is presented in the table below as “MMT-Protected Oligonucleotide Remaining (%)”. MMT-Protected Oligonucleotide Remaining (%) is a measure of the completion of the detritylation reaction, where a value of not more than 0.20% is a passing result. The amount of depurinated oligonucleotide resulting from an undesired acid-mediated degradation side reaction was also analyzed and summarized in the table below as “Depurination (%)”. A Depurination (%) value of not more than 1.5% is a passing result.

TABLE 4
Recovery and purity of large scale detritylation and tangential flow filtration

| | | | | MMT-protected | |
|---|---|---|---|---|---|
| | Reaction | | | Oligonucleotide | |
| | scale | | Recovery | Remaining | Depurination |
| Compound No. | (g) | Process | (%) | (%) | (%) |
| 5-(PO₄)—(CH₂)₆—NH₂ | 3184 | TFF | 93.1% | 0.01 | 0.55 |

1. A method for preparing an oligomeric compound comprising a modified oligonucleotide, comprising:
a) providing a flow filtration system comprising a protected oligomeric compound solution carrying a protected oligomeric compound, wherein the protected oligomeric compound comprises a protected functional group;
b) adding a deprotection reagent to the protected oligomeric compound solution, creating a product solution comprising the deprotected oligomeric compound and a deprotection byproduct, wherein the deprotection byproduct is soluble in the product solution; and
c) circulating the product solution across the semi-permeable membrane for a period sufficient to separate substantially all of the deprotection byproduct from the deprotected oligomeric compound.

2. The method of claim 1, wherein the protected functional group is attached at the 5′-terminal hydroxyl group of the modified oligonucleotide.

3. The method of any one of the preceding claims, wherein the protected functional group is an amine or a hydroxyl.

4. The method of any one of the preceding claims, wherein the protected functional group is a primary amine.

5. The method of any preceding claim, wherein the protected functional group comprises a trityl group.

6. The method of any one of the preceding claims, wherein the trityl group comprises a methoxy.

7. The method of any one of the preceding claims, wherein the trityl group is a 4-monomethoxytrityl or 4-,4′-dimethoxytrityl group.

8. The method of any one of the preceding claims, wherein the trityl group is a 4-monomethoxy trityl (MMT) group.

9. The method of any one of the preceding claims, wherein the oligomeric compound comprises a linker that links the modified oligonucleotide with the protected functional group.

10. The method of claim 9, wherein the linker is an alkyl.

11. The method of claim 10, wherein the alkyl is n-hexyl.

12. The method of any one of the preceding claims, wherein the protected oligomeric compound solution dissolves the protected oligomeric compound.

13. The method of any one of the preceding claims, wherein the deprotection reagent comprises an acid.

14. The method of claim 13, wherein the acid is acetic acid.

15. The method of claim 13, wherein the acid is glacial acetic acid added in 1-2% w/w relative to the volume of the protected oligomeric compound solution.

16. The method of any one of the preceding claims, wherein the product solution has a pH of 3-5.

17. The method of any one of the preceding claims, further comprising heating the product solution.

18. The method of any one of the preceding claims, wherein the product solution is maintained at a temperature of 30-60° C., or about 40° C.

19. The method of any one of the preceding claims, wherein the flow filtration system is a diafiltration system.

20. The method of any one of the preceding claims, wherein circulating the product solution comprises a diafiltration to remove the deprotection byproduct.

21. The method of any one of the preceding claims, wherein diafiltration to remove the deprotection byproduct comprises circulating the product solution against 3 to 20 diavolumes of a diafiltration solution.

22. The method of any one of the preceding claims, wherein the diafiltration solution comprises a salt.

23. The method of any one of the preceding claims, wherein the salt is sodium acetate.

24. The method of any one of the preceding claims, wherein the diafiltration solution comprises an alcohol.

25. The method of claim 24, wherein the alcohol is methanol.

26. The method of any one of the preceding claims, wherein the diafiltration solution comprises 30-90% methanol and 0.01-0.5 M sodium acetate.

27. The method of any one of the preceding claims, wherein the semi-permeable membrane is characterized by a molecular weight cutoff of 1-5 kDa, or about 2 kDa.

28. The method of any one of the preceding claims, further comprising a concentration step in which the protected oligomeric compound solution is partially removed, whereby the concentration of the protected oligomeric compound in the protected oligomeric compound solution is increased.

29. The method of any one of the preceding claims, further comprising a second concentration step in which the product solution is diafiltered against a wash solution comprising an aqueous solution not containing organic solvent.

30. The method of claim 29, wherein the wash solution comprises a salt.

31. The method of claim 30, wherein the salt is sodium acetate, optionally at a concentration of 0.01 to 0.1 M, for example about 0.05 M.

32. The method of any one of claims 29-31, wherein the diafiltration is against 1 to 10 diavolumes of wash solution, optionally about 4 diavolumes.

33. The method of any one of the preceding claims, wherein the concentration step comprises ultrafiltration via the semi-permeable membrane.

34. The method of any one of the preceding claims, wherein the semi-permeable membrane is a cellulose membrane.

35. The method of any one of the preceding claims, wherein recovery of the deprotected oligomeric compound is at least about 85%, 90%, or 95% relative to the amount of protected oligomeric compound.

36. The method of any one of the preceding claims, wherein depurination of the deprotected oligomeric compound is less than about 1% or 0.5%.

37. The method of any one of the preceding claims, wherein at least about 1 kg of deprotected oligomeric compound is recovered.

38. The method of any one of the preceding claims, wherein the modified oligonucleotide consists of 10-30 linked nucleosides, for example 16-23, 16, or 20 linked nucleosides.

39. The method of any one of the preceding claims, wherein the modified oligonucleotide comprises adenine, cytosine, 5-methylcytosine, guanine, thymine, and/or uracil nucleobases, and optionally further comprises one or more hypoxanthine nucleobases.

40. The method of any one of the preceding claims, wherein the modified oligonucleotide comprises a nucleoside sugar moiety selected from the group consisting of 2′-deoxyribosyl sugar moiety, 2′-MOE sugar moiety, LNA sugar moiety, cEt sugar moiety, 2′-NMA sugar moiety, 2′-F sugar moiety, and 2′-OMe sugar moiety, optionally wherein the modified oligonucleotide consists of nucleosides comprising sugar moieties selected from 2′-deoxyribosyl sugar moiety, 2′-MOE sugar moiety, LNA sugar moiety, cEt sugar moiety, 2′-NMA sugar moiety, 2′-F sugar moiety, and 2′-Ome sugar moiety.

41. The method of any one of the preceding claims, wherein the modified oligonucleotide has a gapmer sugar motif.

42. The method of any one of the preceding claims, wherein the modified oligonucleotide comprises a central region of 7-12 nucleosides flanked on the 5′-side by a 5′-external region consisting of 1-6 linked 5′-region nucleosides and on the 3′-side by a 3′-external region consisting of 1-6 linked 3′-region nucleosides; wherein each of the 5′-region nucleosides is a modified nucleoside, and each of the 3′-region nucleosides is a modified nucleoside.

43. The method of any one of the preceding claims, wherein the modified oligonucleotide comprises a central region of 10 nucleosides flanked on the 5′-side by a 5′-external region consisting of 5 linked 5′-region nucleosides and on the 3′-side by a 3′-external region consisting of 5 linked 3′-region nucleosides; wherein each of the 5′-region nucleosides is a modified nucleoside, and each of the 3′-region nucleosides is a modified nucleoside.

44. The method of any one of the preceding claims, wherein the central region comprises linked 2′-β-D-deoxyribosyl nucleosides, each 3′-region nucleoside is selected from a ribo-2′-MOE nucleoside and a cEt nucleoside, and each 5′-region nucleoside is selected from a ribo-2′-MOE nucleoside and a cEt nucleoside.

45. The method of any one of the preceding claims, wherein the modified oligonucleotide comprises or consists of nucleosides comprising sugar moieties selected from 2′-deoxyribosyl sugar moieties, 2′-OMe sugar moieties, and 2′-F sugar moieties, and optionally further comprises one or more 2′-MOE sugar moieties.

46. The method of claim 45, wherein the modified oligonucleotide comprises 2′-OMe nucleosides at one, two, or three of the nucleosides at positions 3, 4, 5 from the 5′-terminus thereof.

47. The method of claim 45 or 46, wherein the modified oligonucleotide comprises 2′-OMe nucleosides at one, two, three, four, or five of the nucleosides at positions 7, 8, 9, 10, and 11 from the 5′-terminus thereof.

48. The method of any one of claims 45 to 47, wherein the modified oligonucleotide comprises 2′-F nucleosides at one, two, or three of the nucleosides at positions 2, 14, and 16 from the 5′-terminus thereof.

49. The method of any one of claims 45 to 48, wherein the modified oligonucleotide comprises 2′-F nucleosides at one, two, or three of the nucleosides at positions 9, 10, and 11 from the 5′-terminus thereof.

50. The method of any one of claims 45 to 49, wherein the 5′-terminal nucleoside of the modified oligonucleotide comprises a stabilized phosphate group.

51. The method of claim 50, wherein the stabilized phosphate group is 5′-vinyl phosphonate, optionally E-5′-vinyl phosphonate.

52. The method of any one of the preceding claims, wherein each nucleoside in the modified oligonucleotide comprises a modified sugar moiety.

53. The method of any one of the preceding claims, wherein the modified oligonucleotide comprises a modified internucleoside linkage.

54. The method of any one of the preceding claims, wherein the modified oligonucleotide comprises a modified internucleoside linkage selected from a phosphorothioate internucleoside linkage and a phosphoramidate internucleoside linkage, optionally wherein the modified oligonucleotide includes only internucleoside linkages selected from phosphorothioate internucleoside linkages, mesyl phosphoramidate internucleoside linkages, and phosphodiester internucleoside linkages.

55. The method of any one of the preceding claims, wherein the modified oligonucleotide comprises a phosphorothioate internucleoside linkage.

56. The method of any one of the preceding claims, wherein the protected oligomeric compound consists of a modified oligonucleotide, a linker, and a protected functional group; optionally wherein the linker is at 3′- or 5′-terminus; for example wherein the linker is a 5′-terminal 6-hexylamino phosphate.

57. The method of any one of the preceding claims, wherein the oligomeric compound comprises a conjugate group or a stabilized phosphate group.

58. The method of any one of the preceding claims, wherein the conjugate group comprises at least one GalNAc moiety, and optionally a triantennary GalNAc cell-targeting moiety.

59. The method of any one of the preceding claims, wherein the conjugate group has the structure:

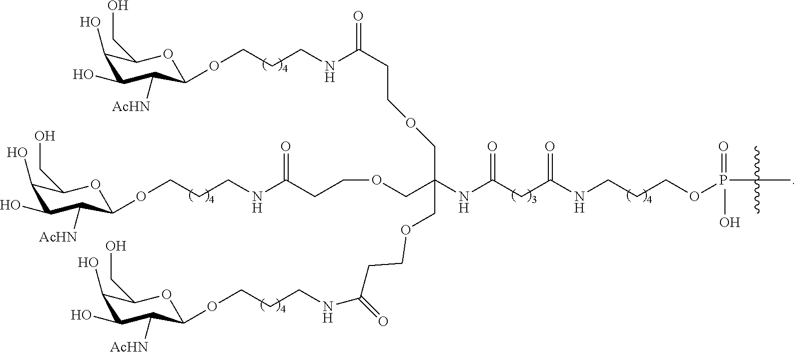

60. An oligomeric compound prepared by the method of any one of the preceding claims.
